(19) United States
(12) Patent Application Publication
(10) Pub. No.: US 2024/0108463 A1
(43) Pub. Date: Apr. 4, 2024

(54) Feature to Improve Fatigue Life and Manage Thrombus in Prosthetic Heart Valves

(71) Applicant: Cephea Valve Technologies, Inc., Abbott Park, IL (US)

(72) Inventors: Jared Floersch, Minneapolis, MN (US); Carlton Jones, Minneapolis, MN (US); Zachary R. Vidlund, Robbinsdale, MN (US)

(73) Assignee: Cephea Valve Technologies, Inc., Abbott Park, IL (US)

(21) Appl. No.: 18/468,946

(22) Filed: Sep. 18, 2023

Related U.S. Application Data
(60) Provisional application No. 63/377,835, filed on Sep. 30, 2022.

(51) Int. Cl. A61F 2/24

(52) U.S. Cl. CPC A61F 2/2418

(57) ABSTRACT

A prosthetic heart valve includes an inner frame having a longitudinal axis, a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis, an outer frame connected to the inner frame, and a sealing feature coupled to the inner frame at a coupling point and extending partially, but not completely, between the coupling point and a point of the outer frame that is radially outward of the coupling point in a direction substantially orthogonal to the longitudinal axis, and wherein in an implanted condition, the sealing feature is positioned such that backpressure from ventricular systole acts on the sealing feature.

### CROSS-REFERENCE TO RELATED APPLICATIONS

The present application claims priority to U.S. Provisional Ser. No. 63/377,835, filed Sep. 30, 2022, the disclosure of which is hereby incorporated by reference in its entirety as if fully set forth herein.

### BACKGROUND OF THE DISCLOSURE

Heart valve disease is a significant cause of morbidity and mortality. A primary treatment of this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after re-expansion.

### BRIEF SUMMARY

One aspect of the disclosure provides a prosthetic heart valve, comprising: an inner frame having a longitudinal axis; a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis; an outer frame connected to the inner frame; and a sealing feature coupled to the inner frame at a coupling point and extending partially, but not completely, between the coupling point and a point of the outer frame that is radially outward of the coupling point in a direction substantially orthogonal to the longitudinal axis, and wherein in an implanted condition, the sealing feature is positioned such that backpressure from ventricular systole acts on the sealing feature.

In one example, the sealing feature comprises a first portion and a second portion.

In one example, the first portion, the second portion, the inner frame, and the outer frame define a partially bounded first pocket that is open and faces a ventricular side of the prosthetic valve.

In one example, the first portion couples with the inner frame at the coupling point, and the second portion couples with the outer frame at a second coupling point.

In one example, the inner frame comprises a first coupling arm extending radially outward of the longitudinal axis and the outer frame comprises a second coupling arm extending radially inward toward the longitudinal axis such that the first coupling arm and the second coupling arm meet and are coupled together at an anchoring point.

In one example, the second coupling point is positioned between an atrial-most point of the outer frame and a point at which the second coupling arm extends from the outer frame.

In one example, the first portion and the second portion each define suture holes for suturing the respective portions.

In one example, the sealing feature further comprises a fourth portion extending radially outward from the second portion, the fourth portion defining suture holes.

In one example, the prosthetic heart valve further includes an embolic retention feature that couples with the outer frame such that a combination of the sealing feature and the embolic retention feature extend completely between the coupling point and the point of the outer frame that is radially outward of the coupling point in the direction substantially orthogonal to the longitudinal axis.

Another aspect of the disclosure provides a prosthetic heart valve, comprising: an inner frame having a longitudinal axis; a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis; an outer frame connected to the inner frame; and a sealing feature including a first portion coupled to the inner frame at a first coupling point, a second portion coupled to the outer frame at a second coupling point, and an embolic retention feature coupled to the outer frame at a third coupling point, wherein the embolic retention feature has a permeability that is greater than at least one of the first portion or the second portion.

In one example, the first portion, the second portion, the inner frame, and the outer frame define a partially bounded first pocket that is open and faces a ventricular side of the prosthetic valve, and the second portion, embolic retention feature, and outer frame define a bounded pocket configured to retain a blood clot therein.

In one example, the embolic retention feature has a permeability that is greater than both of the first portion and the second portion.

In one example, the inner frame comprises a first coupling arm extending radially outward of the longitudinal axis and the outer frame comprises a second coupling arm extending radially inward toward the longitudinal axis such that the first coupling arm and the second coupling arm meet and are coupled together at an anchoring point

In one example, the second coupling point is on a ventricular side of the anchoring point and the third coupling point is on an atrial side of the anchoring point.

In one example, the third coupling point is positioned radially outward relative to the second coupling point.

Another aspect of the disclosure provides a prosthetic heart valve, comprising: an inner frame having a longitudinal axis; a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis; an outer frame; a sealing feature coupled to the inner frame at a first coupling point and extending radially outward and coupling to the outer frame at a second coupling point; and an embolic retention feature coupled to the outer frame at a third coupling point that is distinct from the second coupling point such that a partially bounded pocket is defined and a bounded pocket is defined, wherein the bounded pocket is configured to retain a blood clot therein.

In one example, the sealing feature comprises a first portion and a second portion.

In one example, a combination of the first portion and the embolic retention feature spans an entire annular span between the first coupling point and the third coupling point.

In one example, the embolic retention feature has a greater permeability than both of the first portion and the second portion

In one example, the partially bounded pocket is defined in part by the first portion and the second portion such that the partially bounded pocket is radially inward relative to the bounded pocket.

### BRIEF DESCRIPTION OF THE DRAWINGS

FIG. 1A is a side view of an assembled stent frame of a prosthetic heart valve of the prior art, the stent frame being shown in an expanded condition.

FIG. 1B is a side view of an outer frame of the stent frame of FIG. 1A.

FIG. 1C is a flattened view of the outer stent of FIG. 1B, as if cut longitudinally and laid out flat on a table in an unexpanded condition.

FIG. 1D is a side view of an inner frame of the stent frame of FIG. 1A.

FIG. 1E is a flattened view of the inner stent of FIG. 1D, as if cut longitudinally and laid out flat on a table in an unexpanded condition.

FIG. 2A is a side view of an assembled stent frame of a prosthetic heart valve according to an embodiment of the disclosure, the stent frame being shown in an expanded condition.

FIG. 2B is a side view of an outer frame of the stent frame of FIG. 2A.

FIG. 2C is a flattened view of the outer stent of FIG. 2B, as if cut longitudinally and laid out flat on a table in an unexpanded condition.

FIG. 2D is a side view of an inner frame of the stent frame of FIG. 2A.

FIG. 2E is a flattened view of the inner stent of FIG. 2D, as if cut longitudinally and laid out flat on a table in an unexpanded condition.

FIG. 3A is a cross-sectional schematic diagram of a prosthetic heart valve in the expanded condition and implanted relative to the atrium and ventricle.

FIG. 3B is a cross-sectional schematic diagram of a prosthetic heart valve in the expanded condition and implanted relative to the atrium and ventricle.

FIG. 3C is a cross-sectional schematic diagram of a prosthetic heart valve in the expanded condition and implanted relative to the atrium and ventricle.

FIG. 3D is a cross-sectional sectional schematic diagram of a prosthetic heart valve in the expanded condition and implanted relative to the atrium and ventricle.

FIG. 3E is a cross-sectional sectional schematic diagram of a prosthetic heart valve in the expanded condition and implanted relative to the atrium and ventricle.

FIG. 4 illustrates a partial cross-sectional view of a prosthetic valve in the expanded condition.

FIGS. 5A-C illustrate perspective views of prosthetic heart valves.

### DETAILED DESCRIPTION

As used herein, the term “inflow end,” when used in connection with a prosthetic heart valve, refers to an end of the prosthetic heart valve into which blood first flows when the prosthetic heart valve is implanted in an intended position and orientation. On the other hand, the term “outflow end,” when used in connection with a prosthetic heart valve, refers to the end of the prosthetic heart valve through which blood exits when the prosthetic heart valve is implanted in an intended position and orientation. In the figures, like numbers refer to like or identical parts. As used herein, the terms “substantially,” “generally,” “approximately,” and “about” are intended to mean that slight deviations from absolute are included within the scope of the term so modified. When ranges of values are described herein, those ranges are intended to include sub-ranges. For example, a recited range of 1 to 10 includes 2, 5, 7, and other single values, as well as all sub-ranges within the range, such as 2 to 6, 3 to 9, 4 to 5, and others.

The present disclosure is generally directed to collapsible prosthetic mitral valves, and in particular various features of stents thereof to provide enhanced functionality. However, it should be understood that the features described herein may apply to other types of prosthetic heart valves, including prosthetic heart valves that are adapted for use in other heart valves, such as the tricuspid heart valve. Further, the features of the prosthetic heart valves described herein may, in some circumstances, be suitable for surgical (e.g., non-collapsible) prosthetic heart valves. However, as noted above, the disclosure is provided herein in the context of a collapsible and expandable prosthetic mitral valve.

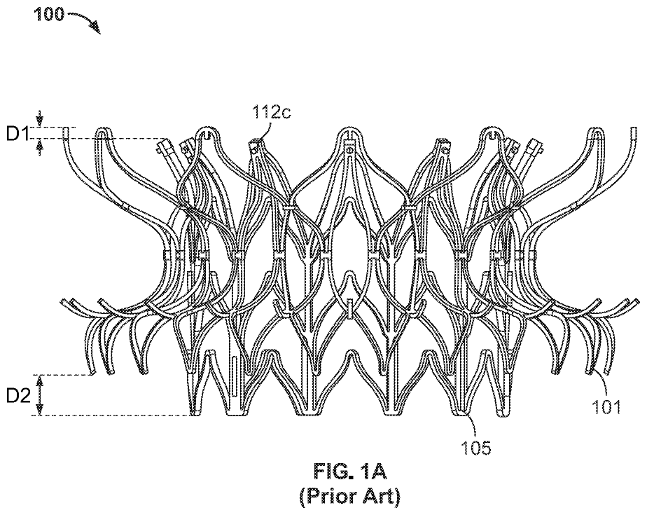

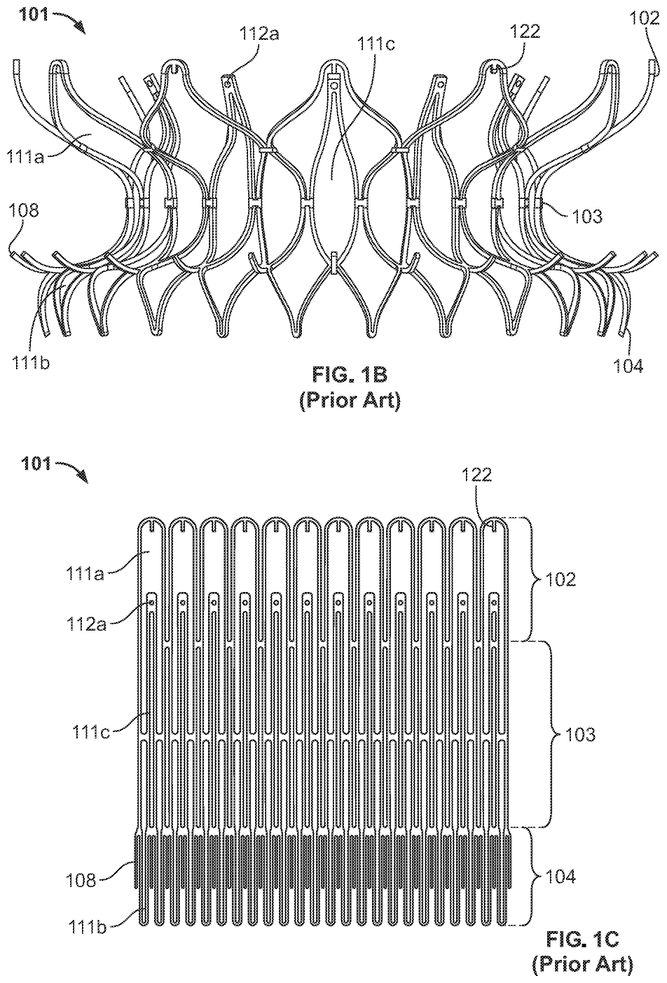

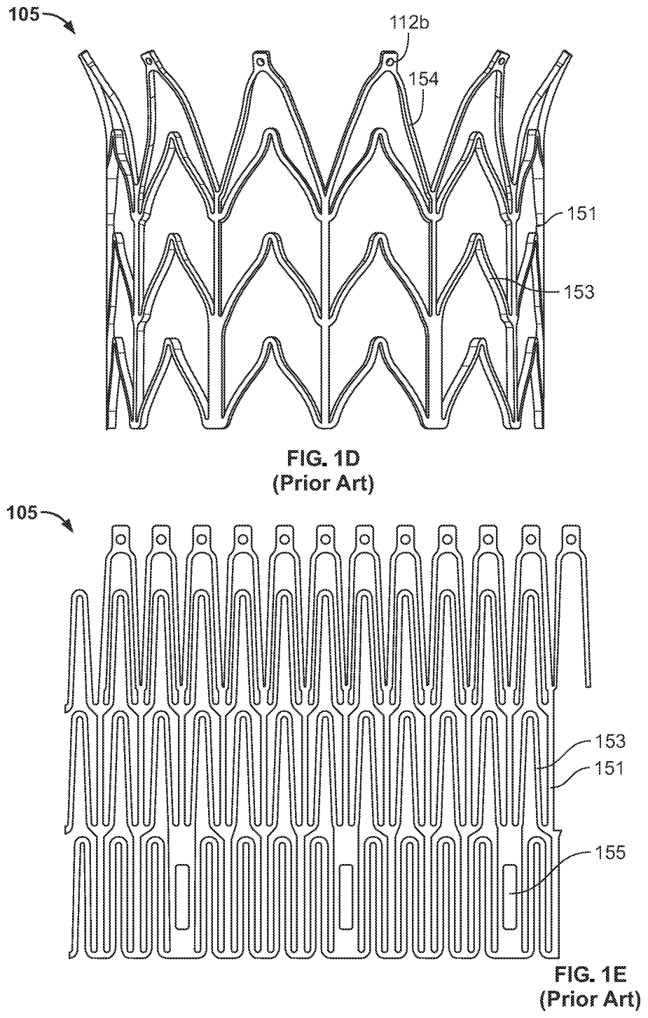

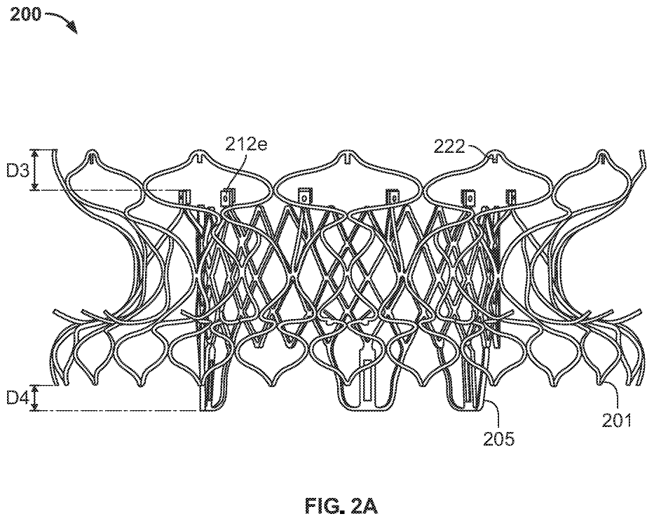

FIG. 1A illustrates an example of a collapsible and expandable prosthetic heart valve 100, according to the prior art, which may be particularly suited for replacement of a native mitral or tricuspid valve. It should be understood that the prosthetic heart valve 100 illustrated in FIG. 1A omits certain features that would typically be included, such as a valve assembly to assist in controlling blood flow through the prosthetic heart valve, and interior and/or exterior fabrics or tissue skirts to assist with providing a seal around the prosthetic heart valve and/or with enhancing tissue ingrowth to fix the prosthetic heart valve within the native heart valve over time. However, for purposes of simplicity, the prosthetic leaflet(s) and skirt(s) are omitted from the drawings for clarity of illustration.

The prosthetic heart valve 100 is illustrated in FIG. 1A in an expanded configuration. The stent of the prosthetic heart valve 100 may include an outer stent or frame 101 and an inner stent or frame 105 positioned radially within the outer frame. The outer frame 101 may be primarily for anchoring the prosthetic heart valve 100 within the native heart valve annulus, while the inner frame 105 may be primarily for holding the prosthetic valve assembly in the desired position and orientation.

Outer frame 101 is illustrated in FIGS. 1B-C isolated from other components of the prosthetic heart valve 100. In FIG. 1B, the outer frame 101 is illustrated in an expanded condition. In FIG. 1C, the outer frame 101 is illustrated in an unexpanded condition, as if cut longitudinally and laid flat on a table. As used herein, the term “unexpanded” refers to the state of the stent prior to being shape-set (e.g., immediately after it is cut from a tube of nitinol). After being formed, the stent may be shape-set to the expanded condition, and may also have a collapsed condition in which the stent is collapsed to a smaller size from its expanded condition. The shape of the stent may be similar, but not necessarily identical, in the unexpanded and collapsed conditions. As shown in FIGS. 1B-C, outer frame 101 may include an atrial portion or anchor 102, a ventricular portion or anchor 104, and a central portion 103 coupling the atrial portion to the ventricular portion. The central portion 103 may be between atrial portion 102 and ventricular portion 104. Atrial portion 102 may be configured and adapted to be disposed on an atrial side of a native valve annulus, and may flare radially outwardly from the central portion 103. Ventricular portion 104 may be configured and adapted to be disposed on a ventricle side of the native valve annulus, and may also flare radially outwardly from the central portion 103. The central portion 103 may be configured to be situated in the valve orifice, for example in contact with the native valve annulus. In use, the atrial portion 102 and ventricle portion 104 effectively clamp the native valve annulus on the atrial and ventricular sides thereof, respectively, holding the prosthetic heart valve 100 in place.

The atrial portion 102 may be formed as a portion of a stent or other support structure that includes or is formed by a plurality of generally diamond-shaped cells, although other suitable cell shapes, such as triangular, quadrilateral, or polygonal may be appropriate. In some examples, the atrial portion 102 may be formed as a braided mesh, as a portion of a unitary stent, or a combination thereof. According to one example, the stent that includes the atrial portion 102 may be laser cut from a tube of nitinol and heat set to a desired shape so that the stent, including atrial portion 102, is collapsible for delivery, and re-expandable to the set-shape during deployment. The atrial portion 102 may be heat set into a suitable shape to conform to the native anatomy of the valve annulus to help provide a seal and/or anchoring between the atrial portion 102 and the native valve annulus. The heat-set atrial portion 102 may be partially or entirely covered by a cuff or skirt, on the luminal and/or abluminal surface of the atrial portion 102. The skirt may be formed of any suitable material, including biomaterials such as bovine pericardium, biocompatible polymers such as ultra-high molecular weight polyethylene, woven polyethylene terephthalate (“PET”) or expanded polytetrafluoroethylene (“ePTFE”), or combinations thereof. The atrial portion 102 may include features for connecting the atrial portion to a delivery system. For example, the atrial portion 102 may include pins or tabs 122 around which sutures (or suture loops) of the delivery system may wrap, so that while the suture loops are wrapped around the pins or tabs 122, the outer frame 101 maintains a connection to the delivery device.

The ventricular portion 104 may also be formed as a portion of a stent or other support structure that includes or is formed of a plurality of diamond-shaped cells, although other suitable cell shapes, such as triangular, quadrilateral, or polygonal may be appropriate. In some examples, the ventricular portion 104 may be formed as a braided mesh, as a portion of a unitary stent, or a combination thereof. According to one example, the stent that includes the ventricular portion 104 may be laser cut from a tube of nitinol and heat set to a desired shape so that the ventricular portion 104 is collapsible for delivery, and re-expandable to the set-shape during deployment. The ventricular portion 104 may be partially or entirely covered by a cuff or skirt, on the luminal and/or abluminal surface of the ventricular portion 104. The skirt may be formed of any suitable material described above in connection with the skirt of atrial portion 102. It should be understood that the atrial portion 102 and ventricular portion 104 may be formed as portions of a single support structure, such as a single stent or braided mesh. However, in other embodiments, the atrial portion 102 and ventricular portion 104 may be formed separately and coupled to one another.

As illustrated in FIG. 1A, the inner frame 105 may be positioned radially within the outer frame 101 when the inner and outer frames are assembled together. Inner frame 105 is illustrated in FIGS. 1D-E isolated from other components of the prosthetic heart valve 100. In FIG. 1D, the inner frame 105 is illustrated in an expanded condition. In FIG. 1E, the inner frame 105 is illustrated in an unexpanded condition, as if cut longitudinally and laid flat on a table. As shown in FIGS. 1D-E, the inner frame 105 may include a plurality of axially or longitudinally extending struts 151 and interconnecting v-shaped strut members 153. According to some embodiments, the inner frame 105 may have more or fewer v-shaped members 153 extending circumferentially around the diameter thereof than the number of cells in the atrial portion 102 and/or ventricular portion 104 of the outer frame 101, such as double or half the number. In some examples, the inner frame 105 may flare radially outwards at the atrial end, e.g., to conform to the flare of the atrial portion 102 of the outer frame 101. One or more prosthetic leaflets may be coupled to the inner frame 105 to form a prosthetic valve assembly, the prosthetic valve assembly configured to allow unidirectional flow of blood through the prosthetic valve assembly from the atrial end toward the ventricular end of the prosthetic heart valve 100. As best illustrated in FIG. 1E, the inner frame 105 may include a plurality of commissure windows 155 formed in axial struts 151. For example, inner frame 105 may include three generally rectangular-shaped commissure windows 155 equidistantly spaced around the circumference of the inner frame, with each commissure window adapted to provide a location for coupling two adjacent prosthetic leaflets to the axial strut 151. However, more or fewer commissure windows 155 may be provided depending on how many prosthetic leaflets will be coupled to the inner frame 105.

The outer frame 101 and/or the inner frame 105 may be formed of a superelastic and/or shape memory material such as nitinol. According to some examples, other biocompatible metals and metal alloys may be suitable. For example, superelastic and/or self-expanding metals other than nitinol may be suitable, while still other metals or metal alloys such as cobalt chromium or stainless steel may be suitable, particularly if the stent or support structure is intended to be balloon expandable. In some examples, the outer frame 101 and/or inner frame 105 may be laser cut from one or more tubes, such as a shape memory metal tube. The shape memory metal tube may be nitinol or any other bio-compatible metal tube. For example, the outer frame 101 may be laser cut from a first tube while the inner frame 105 may be laser cut from a second tube of smaller diameter.

The prosthetic heart valve 100 may be adapted to expand from a collapsed or constrained configuration to an expanded configuration. According to some examples, the prosthetic heart valve 100 may be adapted to self-expand, although the prosthetic heart valve could instead be partially or fully expandable by other mechanisms, such as by balloon expansion. The prosthetic heart valve 100 may be maintained in the collapsed configuration during delivery, for example via one or more overlying sheaths that restrict the valve from expanding. The prosthetic heart valve 100 may be expanded during deployment from the delivery device once the delivery device is positioned within or adjacent to the native valve annulus. In the expanded configuration, the atrial portion 102 and ventricular portion 104 may extend radially outward from a central longitudinal axis of the prosthetic heart valve 100 and/or central portion 103, and may be considered to flare outward relative to the central longitudinal axis of the replacement valve and/or central portion 103. The atrial portion 102 and ventricular portion 104 may be considered flanged relative to central portion 103. The flared configuration of atrial and ventricular portions 102, 104 relative to central portion 103 is described in the context of a side view of the outer frame 101, as can be best seen in FIG. 1B. In some embodiments, the flared configuration of the atrial and ventricular portions 102, 104 and the central portion 103 may define a general hour-glass shape in a side view of the outer frame 101. That is, the atrial and ventricular portions 102, 104 may be flared outwards relative to the central portion 103 and then curved or bent to point at least partially back in the axial direction. It should be understood, however, that an hour-glass configuration is not limited to a symmetrical configuration.

The outer frame 101 may be configured to expand circumferentially (and radially) and foreshorten axially as the prosthetic heart valve 100 expands from the collapsed delivery configuration to the expanded deployed configuration. As described herein, the outer frame 101 may define a plurality of atrial cells 111a in one circumferential row and a plurality of ventricular cells 111b in another circumferential row. Each of the plurality of cells 111a, 111b may be configured to expand circumferentially and foreshorten axially upon expansion of the outer frame 101. As shown, the cells 111a-b may each be diamond-shaped. In the illustrated embodiment, the outer frame 101 includes twelve atrial cells 111a, and twenty-four ventricular cells 111b. In addition, a third plurality of cells 111c may be provided in another circumferential row. Cells 111c may have a first end that is within a corresponding atrial cell 111a, at least when the frame is collapsed (similar to the unexpanded condition shown in FIG. 1C). Cells 111c may have a second end that is positioned between pairs of adjacent ventricular cells 111b, at least when the frame is collapsed (similar to the unexpanded condition shown in FIG. 1C). In this particular example, the outer frame 101 includes twelve center cells 111c.

Still referring to FIGS. 1B-C, a pin or tab 122 may extend from an apex of each atrial cell 111a in a direction toward the outflow end of the outer frame 101. Although one pin or tab 122 is illustrated in each atrial cell 111a, in other embodiments fewer than all of the atrial cells may include a pin or tab. Each center cell 111c may include an aperture 112a or other coupling feature at a first apical end thereof for coupling to the inner frame 105, as is described in greater detail below. In the illustrated embodiment, the aperture 112a is positioned at the inflow apex of center cells 111c, and each center cell includes an aperture, although in other embodiments fewer than all of the cells may include such apertures. In the expanded condition of the outer frame 101, as shown in FIG. 1B, the apex of the center cells 111c that include the apertures 112a may be positioned radially inwardly of the apex of the atrial cells 111a near the inflow end of the outer frame. In addition, each center cell 111c may include a tine or barb 108 extending from the opposite apex on the outflow end of the center cell, although fewer than all of the center cells may include such barbs. In the collapsed condition of the outer frame 101 (similar to the unexpanded condition shown in FIG. 1C), each barb 108 extends toward the outflow end of the outer frame, each barb being positioned between two adjacent ventricular cells 111b. In the expanded condition of the outer frame 101, as shown in FIG. 1B, the barbs 108 may hook upwardly back toward the inflow end, the barbs being configured to pierce native tissue of the valve annulus, such as the native leaflets, to help keep the prosthetic heart valve from migrating under pressure during beating of the heart. Typically, the term “tine” may refer to a structure configured to pierce into tissue, while the term “barb” may refer to a tine that also includes a barb-like structure to prevent the barb from pulling out of the tissue once pierced. However, as used herein, the term “barb” includes tines, with or without actual “barb”-like structures that prevent pulling out of tissue, unless specifically noted otherwise.

The inner frame 105 may be configured to expand circumferentially (and radially) while maintaining the same (or about the same) axial dimension (e.g., be non-foreshortening) as the prosthetic heart valve 100 expands from the collapsed delivery configuration to the expanded configuration. The axial struts 151 may contribute to this non-foreshortening functionality. By being non-foreshortening, the inner frame 105 may prevent (or reduce) strain from being placed on the prosthetic leaflets when the inner frame 105 transitions between the collapsed and expanded conditions. Thus, while the outer frame 101 may be designed to be foreshortening, the inner frame 105 may be designed so as to be substantially non-foreshortening.

Inner frame 105 may include twelve longitudinal struts 151, with three rows of twelve v-shaped members 153. However, in other embodiments, more or fewer longitudinal struts 151 may be included, and more or fewer rows of v-shaped members 153 may be included. In the illustrated embodiment, the number of longitudinal struts 151 is equal to the number of atrial cells 111a of the outer frame 101. In addition, v-shaped coupling members 154 may extend from each adjacent pair of longitudinal struts 151. These v-shaped coupling members 154 may have half-diamond shapes, with the apex of each half-diamond shape including an aperture 112b, the v-shaped coupling members generally flaring radially outwardly in the expanded condition of inner frame 105.

Referring back to FIG. 1A, in the expanded conditions of the outer frame 101 and the inner frame 105, the top portion of the center cells 111c may flare outwardly with a contour that substantially matches the outward flare of the v-shaped coupling members 154, so that apertures 112a and 112b align with each other. A coupling member, such as a rivet 112c, may pass through apertures 112a and 112b to couple the outer frame 101 to the inner frame 105.

Additional features and example replacement valves may be described in International patent application publication WO/2018/136959, filed Jan. 23, 2018, and titled “REPLACEMENT MITRAL VALVES,” which is hereby incorporated by reference herein.

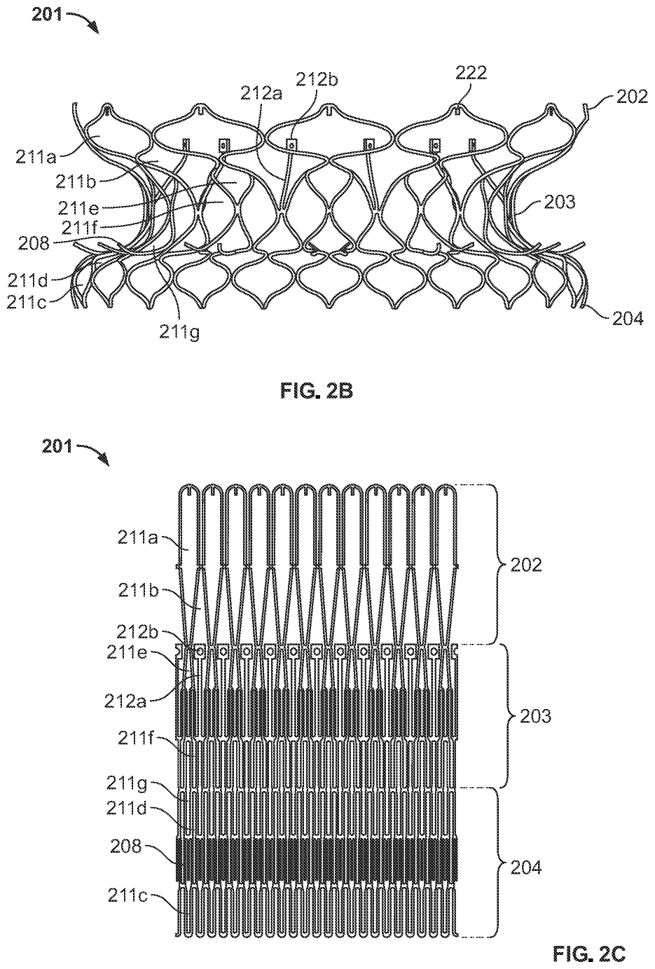

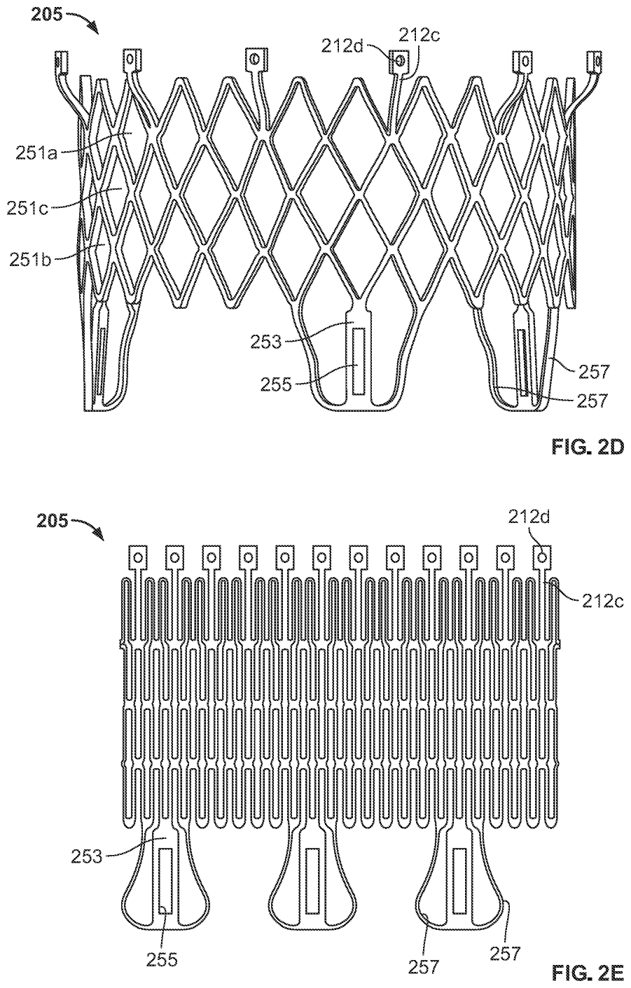

FIG. 2A illustrates another embodiment of a collapsible and expandable prosthetic heart valve 200, which may be particularly suited for replacement of a native mitral or tricuspid valve. The overall general structure of prosthetic heart valve 200 may be substantially similar to that of prosthetic heart valve 100 in both structure and function, but prosthetic heart valve 200 may have various differences to provide for certain benefits compared to prosthetic heart valve 100. For the purpose of brevity, only the differences between prosthetic heart valve 200 compared to prosthetic heart valve 100 are described in detail below, with the remaining features of prosthetic heart valve 200 being similar or identical to the corresponding features of prosthetic heart valve 100. As with prosthetic heart valve 100, it should be understood that the prosthetic heart valve 200 illustrated in FIG. 2A omits certain features such as prosthetic leaflets and luminal and/or abluminal stent skirts. The prosthetic heart valve 200 is illustrated in FIG. 2A in an expanded configuration. The stent of the prosthetic heart valve 200 may include an outer stent or frame 201 and an inner stent or frame 205 positioned radially within the outer frame.

Outer frame 201 is illustrated in FIGS. 2B-C isolated from other components of the prosthetic heart valve 200. In FIG. 2B, the outer frame 201 is illustrated in an expanded condition. In FIG. 2C, the outer frame 201 is illustrated in an unexpanded condition, as if cut longitudinally and laid flat on a table. Similar to outer frame 101, outer frame 201 may include an atrial portion or anchor 202, a ventricular portion or anchor 204, and a central portion 203 coupling the atrial portion to the ventricular portion.

The outer frame 201 may be configured to expand circumferentially (and radially) and foreshorten axially as the prosthetic heart valve 200 expands from the collapsed delivery configuration to the expanded deployed configuration. The outer frame 201 may define a plurality of atrial cells 211a, 211b in two circumferential rows. For example, the first row of atrial cells 211a may be generally diamond shaped and positioned on the inflow end of the outer frame 201. The second row of atrial cells 211b may be positioned at least partially between adjacent atrial cells 211a in the first row, with the atrial cells 211b in the second row being positioned farther from the inflow end than the first row of atrial cells 211a. The outer stent 201 may include twelve atrial cells 211a in the first row each having a diamond-shape, and twelve atrial cells 211b in the second row each having a skewed diamond shape. This skewed diamond shape, which is wider nearer the inflow (or top) end and narrower nearer the outflow (or bottom) end, may assist in transitioning from twelve cells per row on the atrial side of the stent to twenty-four cells per row on the ventricular side.

The outer frame 201 may include a plurality of ventricular cells 111c in a first row, and another plurality of ventricular cells 11d in a second row. The first row of ventricular cells 211c may be at the outflow end of the outer frame 201, and the second row of ventricular cells 211d may be positioned farther from the outflow end than, and adjacent to, the first row of ventricular cells 211c. In the illustrated embodiment the first and second rows of ventricular cells 211c, 211d are all generally diamond-shaped and have substantially the same, or an identical, size, with twenty-four cells in the first row of ventricular cells 211c and twenty-four cells in the second row of ventricular cells 211d.

Outer stent 201 is also illustrated as including three rows of center cells. A first row of center cells 211e is positioned adjacent the atrial end of the outer stent 201, each cell 211e being positioned between a pair of adjacent atrial cells 211b. Each center cell 211e may be substantially diamond-shaped, but it should be understood that adjacent center cells 211e do not directly touch one another. The first row of center cells 211e may include twelve center cells 211e, with the combination of atrial cells 211b and the center cells 211e helping transition from rows of twelve cells on the atrial side to rows of twenty-four cells on the ventricular side. A second row of center cells 211f may be positioned at a longitudinal center of the outer frame 201, each center cell 211f being positioned between an atrial cell 211b and center cell 211e. In the illustrated embodiment, center cells 211f in the second row may be diamond-shaped, with the second row including twenty-four center cells 211f. Finally, a third row of center cells 211g may be positioned between the second row of center cells 211f and the second row of ventricular cells 211d. The third row of center cells 211g may include twenty-four cells and they may each be substantially diamond-shaped.

All of the cells 211a-g may be configured to expand circumferentially and foreshorten axially upon expansion of the outer frame 201. Similar to outer frame 100, a pin or tab 222 may extend from an apex of each atrial cell 211a in the first row in a direction toward the outflow end of the outer frame 201. Although one pin or tab 222 is illustrated in each atrial cell 211a in the first row, in other embodiments fewer than all of the atrial cells in the first row may include a pin or tab. Whereas outer frame 101 included apertures 112a at an apex of a center cell 111c, outer frame 201 may instead include coupling arms 212a. Each coupling arm 212a may be a strut that is coupled to a bottom or outflow apex of each atrial cell 211b in the second row, with each strut extending toward the inflow end of the outer frame 201 to a free end of the coupling arm 212a. The free end of each coupling arm 212a may include an aperture 212b for coupling to the inner frame 205, as described in greater detail below. In the collapsed condition (similar to the unexpanded condition shown in FIG. 2C), each coupling arm 212a is substantially surrounded by an atrial cell 211b in the second row. In the expanded condition, best shown in FIG. 2B, the coupling arms 212a may extend radially inwardly and have a contour so that the free end extends substantially parallel to the center longitudinal axis of the outer frame 201. In addition, outer frame 201 may include a plurality of tines or barbs 208 extending from a center portion or ventricular portion of the outer frame for piercing native tissue in the native annulus or in the native leaflets. In the illustrated embodiment, each barb 208 is connected to a ventricular cell 211d in the second row. In some embodiments, the barb 208 may be coupled to an inflow or outflow apex of each cell. In the particular illustrated embodiment, the barbs 208 are couple to ventricular cells 211d on an inflow half of the cell, on either side of the inflow apex. For example, the barb 208 in one ventricular cell 211d may be coupled to the inflow half of that cell on a right side of the apex, with the adjacent ventricular cell 211d having a barb coupled to the inflow half of that cell on a left side of the apex. With this configuration, the barbs 208 are provided in pairs with relatively little space between the barbs of a pair, but a relatively large space between adjacent pairs. However, it should be understood that the barbs 208 may in other embodiments be centered with even spacing between adjacent barbs, similar to that shown and described in connection with FIG. 1B. In the collapsed condition of the outer frame 201 (similar to the unexpanded condition shown in FIG. 2C), each barb 208 extends toward the outflow end of the outer frame, each barb being positioned within a ventricular cell 211d in the second row. In the expanded condition of the outer frame 201, as shown in FIG. 2B, the barbs 208 may hook upwardly back toward the inflow end, the barbs being configured to pierce native tissue of the valve annulus, such as the native leaflets, to help keep the prosthetic heart valve from migrating under pressure during beating of the heart. In some embodiments, each coupling arm may be provided as a pair of coupling arms. For example, the disclosure provided herein related to sealing features and/or embolic retention features may be used in combination with frames similar or identical to those described in U.S. Provisional Patent Application No. 63/484,017, titled “Prosthetic Heart Valve Frame with Double-Arm Connection” and filed on Feb. 9, 2023, the disclosure of which is hereby incorporated by reference herein.

As illustrated in FIG. 2A, the inner frame 205 may be positioned radially within the outer frame 201 when the inner and outer frames are assembled together. Inner frame 205 is illustrated in FIGS. 2D-E isolated from other components of the prosthetic heart valve 200. In FIG. 2D, the inner frame 205 is illustrated in an expanded condition. In FIG. 2E, the inner frame 205 is illustrated in an unexpanded condition, as if cut longitudinally and laid flat on a table. Whereas inner frame 105 includes longitudinal struts 151 and is non-foreshortening, inner frame 205 instead includes a plurality of rows of diamond-shaped cells so that the inner frame 205 foreshortens upon expansion. In the illustrated example, inner frame 205 includes three rows of diamond-shaped cells, including a first row of cells 251a at the inflow end of the inner frame, a second row of cells 251b at the outflow end of the inner frame, and a third row of cells 251c positioned between the first and second rows. In some embodiments, the inner frame 205 may include more or fewer rows of cells. In the expanded condition shown in FIG. 2D, the three rows of cells 251a-c may be substantially cylindrical.

One or more prosthetic leaflets may be coupled to the inner frame 205 to form a prosthetic valve assembly, the prosthetic valve assembly configured to allow unidirectional flow of blood through the prosthetic valve assembly from the atrial end toward the ventricular end of the prosthetic heart valve 200. As illustrated in FIGS. 2D-E, the inner frame 205 may include a plurality of commissure windows 255 formed in axial struts 253 extending from selected cells 251b at the outflow end of the inner frame 205. For example, inner frame 205 may include three generally rectangular shaped commissure windows 255 equidistantly spaced around the circumference of the inner frame, with each commissure window adapted to provide a location for coupling two adjacent prosthetic leaflets to the axial strut 253. However, more or fewer commissure windows 255 may be provided depending on how many prosthetic leaflets will be coupled to the inner frame 205. Additional support struts 257 may connect the axial struts 253 to the cells 251b. In particular, a first support strut 257 may couple the outflow end of each axial strut 253 to the outflow apex of a first cell 251b on a first side of the axial strut, and a second support strut 257 may couple the outflow end of each axial strut 253 to the outflow apex of a second cell 251b on a second opposite side of the axial strut, with the axial strut coupled to a third cell 251b between the first and second cells. As shown in FIGS. 2D-E, the support struts 257 may be contoured so as to avoid presenting any sharp tips, which may help avoid damaging the anatomy.

The inner frame 205 may also include a plurality of coupling arms 212c. Each coupling arm 212c may have a first end coupled to the inner frame 205 at an inflow end of the inner frame. In particular, the first end of each coupling arm 212c may be attached to a junction between two adjacent cells 251a in the first row at the inflow end. The coupling arms 212c may extend in a direction away from the outflow end of the inner frame 205 to a free end, with the free end including an aperture 212d therein. In the expanded condition, as shown in FIG. 2D, the coupling arms 212c may initially extend radially outwardly from the inner frame 205, with the free end being contoured so that the free end extends substantially parallel to the longitudinal axis of the inner frame 205. In the illustrated embodiment, inner frame 205 may include a total of twelve coupling arms 212c spaced equidistantly around the circumference of the inner frame. Preferably, the number of coupling arms 212c corresponds to the number of coupling arms 212a. Referring back to FIG. 2A, a coupling member, such as a suture or a rivet 212e, may pass through apertures 212b and 212d to couple the outer frame 201 to the inner frame 205.

Various differences between prosthetic heart valves 100 and 200 are now described in greater detail. Outer frame 101 may include a relatively small number of cells which each define a relatively large area. For example, outer frame 101 includes only two rows of cells 111a, 111b for anchoring (which excludes the row of cells 111c which in large part serve to connect the outer frame 101 to the inner frame 105). On the other hand, despite having a generally similar profile as outer frame 101, outer frame 201 includes a larger number of cells that typically define a smaller area. For example, outer frame 201 may be thought of as including six rows of full cells (if cells 211b and 211e are counted as a single row considering the circumferential overlap between those cells). One result of this difference is that the outer frame 101 may have relatively little redundancy compared to outer frame 201. Thus, in the event that a strut defining a cell (or a portion thereof) fractures, the likelihood of stent failure (or the likely detrimental effect of a failure) may be significantly reduced in outer stent 201 compared to outer stent 101, due to increased redundancy in the design. Another benefit of the increased number of cells in outer stent 201 compared to outer stent 101 concerns any tissue and/or fabric skirts coupled to the outer stent 201. For example, due to additional stent structure, more options may be available for how and where to attach tissue and/or fabric skirts to the outer stent 201. This additional stent structure may also better distribute the pressure applied by outer stent 201 to the patient's tissue, thereby reducing the risk of tissue erosion. Still further, the greater number of cells, and smaller area of cells, of outer stent 201 compared to outer stent 101, may allow for reduced forces required to collapse the outer stent 201 during loading into a delivery device, and also reduce forces experienced during deploying the outer stent. This may result in lower strain experienced by the outer stent 201, compared to outer stent 101, and thus improve durability of the outer stent 201. Stated in another way, having more cells with a desired aspect ratio may allow for each cell to have a relatively small strut width, while still being able to maintain a desired stiffness. The diamond cell pattern may allow for the cells (and the stent) to collapse without significant twisting or torsion. This type of twisting or torsion, which may be a primary driver of higher strain, may be more likely to occur in outer stent 101 compared to outer stent 201. The smaller strut width and reduced twisting may provide lower strains, allows sheathing to smaller diameters and improvements in durability

There are various additional differences between prosthetic heart valves 100 and 200, including between the inner stents 105 and 205. For example, inner stent 105 includes commissure windows 155 in axial struts 151 that form part of the outflow end of the inner stent 105, whereas the commissure windows 255 of inner stent 205 extend beyond the main body of the remainder of the inner stent 205. This may allow the main body of inner stent 205 to be shorter than the main body of inner stent 105. In turn, the inner frame 205 may be able to extend a distance D4 beyond the outflow end of the outer stent 201 (see FIG. 2A) that is smaller than the distance D2 that the inner frame 105 extends beyond the outflow end of the outer stent 101 (see FIG. 1A). This may be desirable because, if there is less structure extending into the left (or right) ventricle, there is a smaller likelihood that the inner stent 205 will obstruct the left (or right) ventricular outflow tract, compared to inner stent 105. In other words, there is little or no structure between adjacent commissure windows 255 that would obstruct blood flow, while there is a relatively larger amount of stent structure between adjacent commissure windows 155. Additionally, during ventricular systole, there is relatively small amount of stent structure blocking blood from pressing against the outflow end of the prosthetic leaflets, meaning that the prosthetic leaflets may be faster to coapt with each other during ventricular systole as a result of the extension of the commissure windows 255 farther than adjacent stent structure. The shorter distance of the main body of the inner stent 205 may also provide for greater maneuverability of prosthetic heart valve 200 compared to prosthetic heart valve 100 during deployment and/or repositioning of the prosthetic heart valve. However, the design of inner stent 205 may result in the commissure windows 255 being more likely to deflect during use compared to commissure windows 155 of inner stent 105. In other words, commissure windows 255 may be more cantilevered than commissure windows 155. Thus, when the prosthetic valve assembly is under pressure, particularly when the prosthetic leaflets are closed and are resisting retrograde blood flow, the commissure windows 255 may have a greater tendency to deflect inwardly compared to commissure windows 155. To mitigate this possibility, the commissure windows 255 may include additional supports, in the form of support struts 257 described above, to form a “webbed commissure” structure. This “webbed commissure” structure may also provide a relatively atraumatic structure at the commissure windows 255, which may help avoid piercing any native tissue. Thus, the design of inner stent 205, including that of the commissure windows 255 and the support struts 257, allows for a relatively small protrusion of the inner stent 205 into the ventricle while also helping achieve optimal deflection of the commissure windows 255. These webbed commissures may also provide additional benefits to leaflet closure due to better fluid access to the free margin of the leaflets and a more robust sewing pattern at the commissures such that a metal retaining plate is not required and durability is improved.

Another difference between inner frame 105 and 205 is the position and structure of the v-shaped coupling members 154 (which include coupling aperture 112b) compared to coupling arms 212c (which include coupling aperture 212d). For example, v-shaped coupling members 154 are coupled to the main body of the inner frame 105 at two locations (the two struts that form the “v”-shape), whereas coupling arms 212c are coupled to the main body of inner frame 205 at only one location. This coupling at one location may be robust from a durability standpoint, while also avoiding twisting of struts during expansion and/or shape-setting. For relatively large sized prosthetic heart valves (which include relatively large frames), this may be especially important because the distance between the inner and outer frames may be relatively large. Further, coupling arms 212c may be coupled to inner frame 205 so that aperture 212d extends beyond the inflow end of the inner frame 205 a distance (see FIG. 2A) that is smaller than the distance which aperture 112b extends beyond the inflow end of the inner frame 105 (see FIG. 1A). At least partially as a result of this, the inflow end of the outer frame 201 extends a distance D3 beyond the location of the coupling rivets 212e (see FIG. 2A) that is larger than the distance D1 which the inflow end of the outer frame 101 extends beyond the location of the coupling rivets 112c (see FIG. 1A). Release from the delivery system may be highly dependent on the angle of the pin or tab 222 relative to the axis and the distance D3. A central lumen that connects all the suture loops is pushed downward to release the suture loops. The angle from the pin or tab 222 to the interfering inner frame 205 may be important for release. The larger the angle, the easier the release. Thus, as can be best seen by comparing FIG. 2A to FIG. 1A, there is a relatively large amount of clearance in the atrial cells 211a around pin or tab 222 compared to the amount of clearance in the atrial cells 111a around pin or tab 122. As noted above, during deployment of the prosthetic heart valve 200, sutures or suture loops may loop around pins or tabs 222 to maintain a physical connection between the prosthetic heart valve and the delivery device. After deployment of the prosthetic heart valve 200, the suture loops may be advanced to slip the suture loops off the pins or tabs 222 to fully disconnect the prosthetic heart valve 200 from the delivery device. The larger amount of clearance around the pins or tabs 222, compared to the amount of clearance around pins or tabs 122, may make this process easier and reduce the likelihood of the sutures or suture loops failing to disconnect from the pins or tabs 222, for example via obstruction with other nearby stent structure.

For each of the frames 101, 105, 201, 205 described above, the wall thickness of each individual stent may be substantially constant, whether or not the inner frames 105, 205, have the same wall thicknesses as the corresponding outer frames 101, 201. However, in some embodiments, the stents that form the prosthetic heart valve 200 may have varying wall thickness. For example, the wall thickness of the outer stent 201 near the inflow end and/or near the outflow end may be reduced relative to the wall thickness of the remainder of the outer stent to reduce the stiffness of the atrial and/or ventricular tips of the outer stent 201 to reduce the likelihood of causing trauma to the tissue. For example, the wall thickness of the outer stent in the atrial cells 211a of the first row may be reduced compared to the remainder of the outer stent 201. In one example, only about half the atrial cells 211a in the first row, for example the inflow half, may have a reduced wall thickness compared to the remainder of the outer stent 201. The decrease in stent wall thickness may be gradual or abrupt, for example via a step change. The decreased thickness areas of the outer stent 201 may be created by any suitable method, including for example forming the outer stent 201 of a constant thickness, and then grinding down the stent to a smaller thickness at the desired locations. Additionally, or alternatively, the ventricular cells 211c in the first row may have a reduced wall thickness compared to the remainder of the outer stent 201. As with atrial cells 211a, the wall thickness of ventricular cells 211c may be reduced either gradually or abruptly, including at about half the length of the ventricular cells 211c on the outflow portion of those ventricular cells 211c. With this configuration, one or both tip ends of the outer stent 201 may be provided with reduced thickness to provide reduced stiffness relative to other portions of the outer stent 201 in order to reduce trauma to native tissue. It should further be noted that the atrial and ventricular tip ends of the outer stent 201 may be generally low strain locations compared to the other portions of the outer stent 201. As a result, reducing the stent wall thickness at these locations may not substantially hinder durability of the stent.

In addition or alternatively to reducing the stent wall thickness at one or both tip ends of the outer stent 201, the stent wall thickness near the central waist portion of the outer stent 201 may be increased relative to other portions of the outer stent 201 in order to increase stiffness in this area. For example, the second row of center cells 211f may have a stent wall thickness that is larger than immediately adjacent areas of the stent body 201. For example a portion of the center cells 211f, or the entire center cells 211f, which may include portions of adjacent cells 211b, 211e, 211g, may have a wall thickness greater than all remaining portions of the outer stent 201. When prosthetic heart valve 200 is implanted into a native valve annulus, as noted above, this center portion may be in contact with the native valve annulus while the atrial and ventricular ends wrap around the native valve annulus. As a result, when the heart contracts to pump blood, the center waist portion of the outer stent 201 may be subjected to a relatively large amount of contractile forces. While it may be desirable for the outer stent 201 to have some level of flexibility so as conform to the shape of the native valve annulus, it is typically not desirable for forces imparted on the outer stent 201 to transfer to the inner stent 205 and affect the prosthetic valve leaflets therein. Thus, by increasing stent wall thickness in the waist area of the outer stent 201, deformation of the outer stent 201 may be reduced during beating of the heart, which may help reduce any resulting deformation of the inner stent 205 and prosthetic leaflets positioned therein. The stent wall thickness of the waist portion of the outer stent 201 may be increased by any desired method. For example, the entire outer stent 201 may be formed with a constant thickness that is equal to the desired thickness of the waist portion, and the remaining areas of the outer stent 201 may be ground down to a smaller stent wall thickness. In other embodiments, the waist portion of outer stent 201 may be subsequently increased after the outer stent is formed having a constant stent wall thickness, for example by additive manufacturing, spray coating, dip coating, or any other suitable modalities.

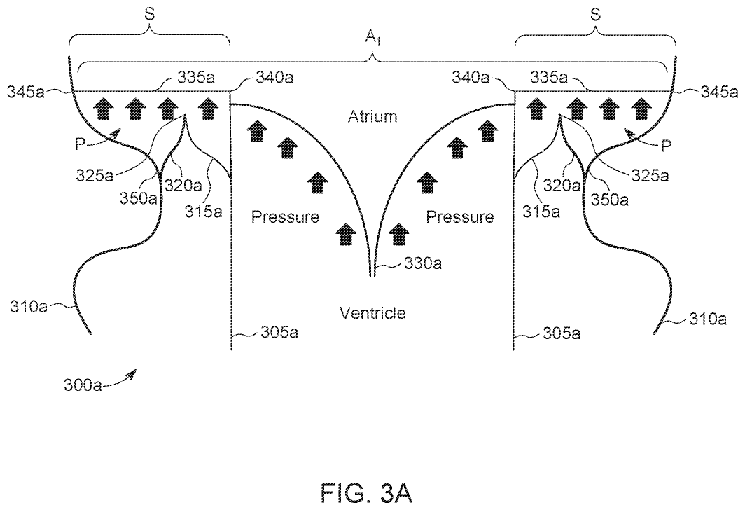

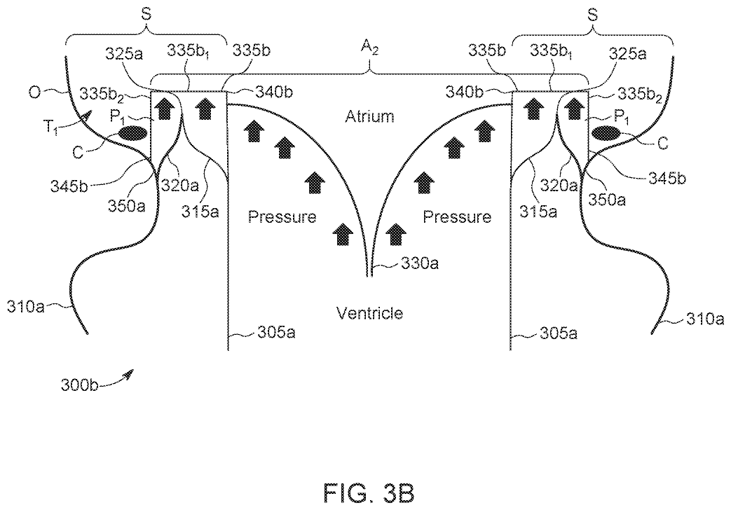

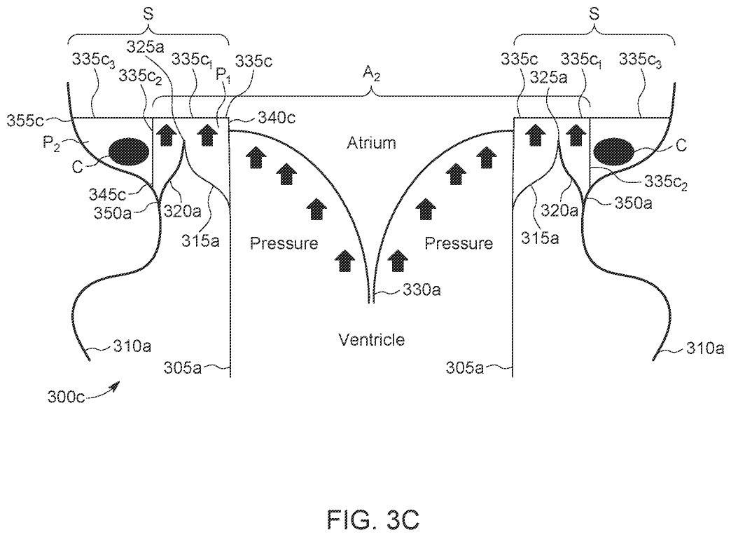

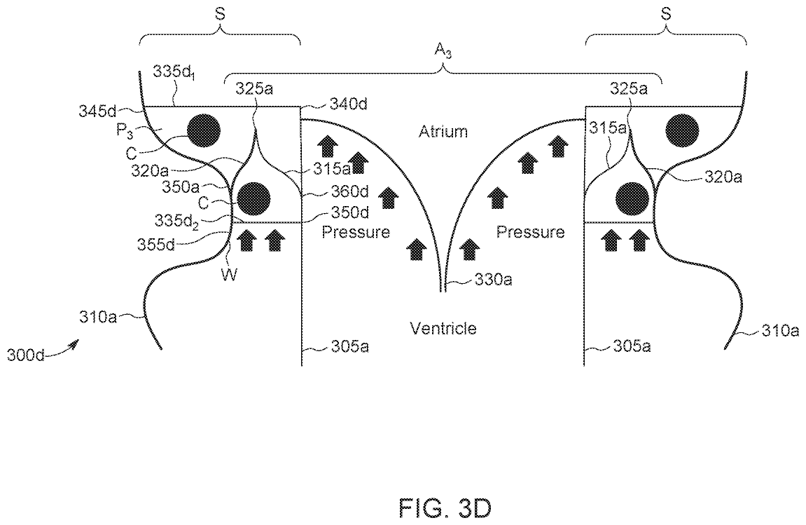

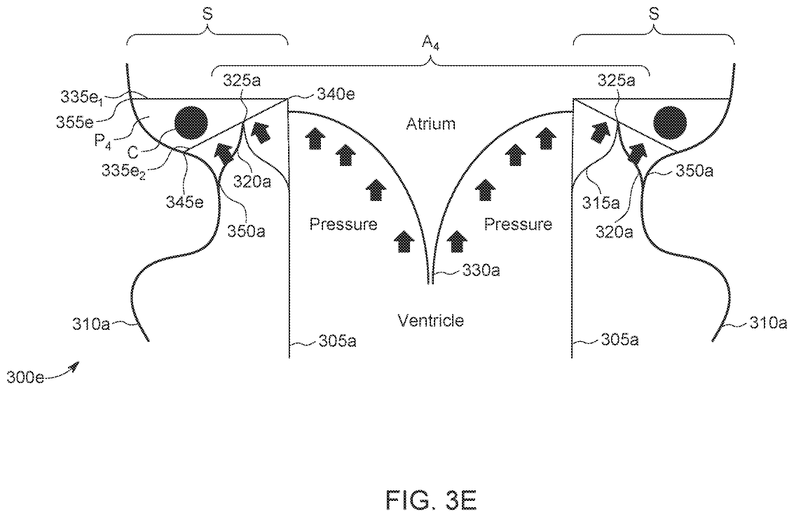

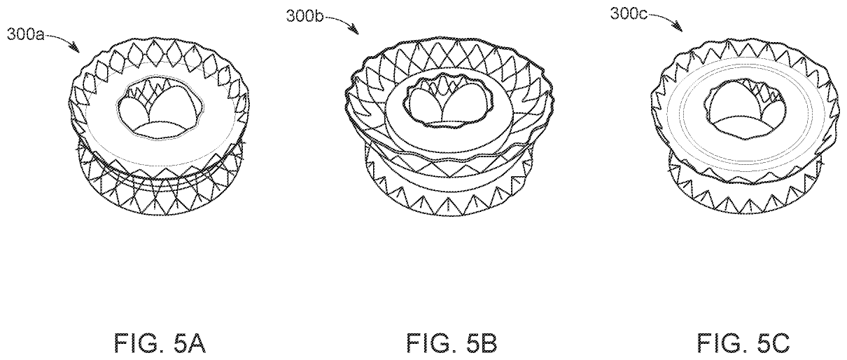

FIG. 3A is a cross-sectional schematic diagram of a prosthetic heart valve 300a in the expanded condition and implanted relative to the atrium and ventricle. FIG. 5A is a perspective view of the prosthetic heart valve 300a of FIG. 3A.

As shown, the prosthetic heart valve 300a can include an inner frame 305a (e.g., inner frame 105 or inner frame 205 described above) positioned radially within an outer frame 310a (e.g., outer frame 101 or outer frame 201). The inner frame 305a can be coupled with the outer frame 310a by any type of structural engagement. In one example, the inner frame 305a includes at least one coupling arm 315a that can be coupled to at least one respective coupling arm 320a of the outer frame 310a at an anchoring point 325a such that the inner frame 305a is anchored to the outer frame 310a on an atrial side of the outer frame 310a. In some examples, coupling arms 315a may be generally similar or identical to coupling arms 212c, and coupling arms 320a may be generally similar or identical to coupling arms 212a. However, other specific implementations of coupling arms 315a, 320a may be suitable for use with prosthetic heart valve 300a. For example, other exemplary coupling configurations are described in U.S. patent application Ser. No. 17/712,290 to Bergin, filed Apr. 4, 2022, entitled Frame Features for Transcatheter Mitral Valve Replacement Device, the entirety of which is hereby incorporated by reference.

The prosthetic heart valve 300a may also include a plurality of prosthetic leaflets 330a (e.g., tissue leaflets) coupled to the inner frame 305a. The plurality of leaflets 330a are movable between an open configuration (not shown) and a closed configuration (as shown in FIG. 3A) in which the leaflets 330a coapt, or meet in sealing abutment. In some embodiments, the prosthetic heart valve 300a includes three prosthetic leaflets 330a, with each pair of adjacent leaflets being coupled to each other and to the inner frame 305a at a commissure feature, such as a feature like commissure window 155 or 255.

For the leaflets 330a to allow unidirectional flow of blood through the prosthetic valve heart valve 300a from the atrial end toward the ventricular end of the prosthetic heart valve 300a, a two-dimensional annular span (represented as S in FIG. 3A) between the inner frame 305a and the outer frame 310a is covered with a sealing feature 335a (also referred to as a sealing member). In this regard, the sealing feature 335a can extend or span an entire radial distance, in a direction substantially orthogonal to the longitudinal axis of the prosthetic heart valve 300a, between coupling point 340a of inner frame 305a and coupling point 345a of outer frame 310a. The sealing feature 335a can be in the shape of a two-dimensional annulus, thereby defining an inner perimeter near the inner frame 305a and an outer perimeter near the outer frame 310a.

The sealing feature 335a can couple with both the inner frame 305a and the outer frame 310a. The sealing feature 335a may couple with the inner frame 305a at any desired portion of the inner frame 305a, and in one example couples at a coupling point 340a that is the atrial-most point of the inner frame 305a. In this regard, the coupling point 340a is positioned radially inward, and on an atrial side, of an anchoring point 325a at which inner frame 305a couples with outer frame 310a. In another example, the coupling point 340a is any point on the inner frame 305a that is on an atrial side of anchoring point 325a, thereby providing the sealing feature 335a clearance with respect to the anchoring point 325a.

The sealing feature 335a may couple with the outer frame 310a at any desired portion of the outer frame 310a, and in one example couples at a coupling point 345a that is between the atrial-most point of the outer frame 310a and a point 350a from which coupling arm 325a extends inward from outer frame 310a. In this regard, the coupling point 345a may be positioned radially outward with respect to coupling point 340a. Further, the coupling point 345a may be positioned both radially outward, and on an atrial side, of an anchoring point 325a at which inner frame 305a couples with outer frame 310a. In the illustrated embodiment, the sealing feature 335a extends in a direction that is generally orthogonal to the direction of blood flow, but in other embodiments, the sealing feature 335 may extend at an oblique angle relative to the direction of blood flow.

The sealing feature 335a can extend and cover the entire two-dimensional annular span S between the coupling point 340a at inner frame 305a and the coupling point 345a at outer frame 310a, thereby defining a partially bounded three-dimensional annular pocket (represented as P in FIG. 3A). The partially bounded three-dimensional annular pocket P is bounded in part by inner frame 305a (including any cuffs or skirts on the inner frame), outer frame 310a (including any cuffs or skirts on the outer frame), and sealing feature 335a. As depicted in FIG. 3A, the partially bounded three-dimensional annular pocket P may also be partially bounded by coupling arm 315a and coupling arm 320a at certain locations, as the coupling arm 315 and coupling arm 320a are arranged in a spaced apart manner around a circumference of the prosthetic heart valve 300a. The pocket P is partially bounded insofar as the pocket P is relatively open on a ventricular or outflow side between the inner frame 305a and the portion of outer frame 310a of least diameter (which in the illustrated embodiment is a central waist of the outer frame 310a).

The sealing feature 335a can be formed at least in part, or in its entirety, of any suitable material that is substantially impermeable to blood and thereby prevents blood flow across the prosthetic heart valve 300a and through the sealing feature 335a when the prosthetic leaflets 330a are in the closed or coapted condition. The sealing feature 335a can be formed of a tissue or a fabric, including synthetic fabrics, such as PET, PTFE, etc.

In a typical tricuspid or mitral valve, the configuration of sealing feature 335a results in a large area for backpressure, resulting from ventricular systole, to act against. The relevant area against which pressure acts is shown as cross-sectional area, represented as A₁, in the cross-section of FIG. 3A. This cross-sectional area A₁ is the combination of the cross-sectional area of the leaflets 330a (i.e., area of 2D projection of leaflets onto a plane that is orthogonal to direction of blood flow) in addition to the cross-sectional area of the sealing feature 335a. In other words, because the leaflets 330a and the sealing feature 335a are both substantially impermeable to blood flow, as the leaflets 330a coapt during ventricular systole and the pressure within the ventricle is significantly higher than the pressure within the atrium, both the leaflets 330a and the sealing feature 335a are the surfaces that separate the high pressure of the ventricle from the low pressure of the atrium. In order for the prosthetic heart valve 300a to remain in a stable position within the native valve annulus, the leaflets 330a and sealing feature 335a must resist this pressure gradient. The construction of the outer frame 310a typically results in a very strong fixation of the outer frame 310a to the native valve annulus. However, the inner frame 305a is only coupled to the outer frame 310a via coupling arms 315a, 320a and sealing feature 335a. Thus, the backpressure during ventricular systole may have the most significant effect on the inner frame 305a, tending to move the inner frame 305a slightly in the atrial direction during ventricular systole. The resulting force on the inner frame 305a during ventricular systole is proportional, or equal, to the backpressure multiplied by the area A₁ against which the pressure acts. As noted above, this resulting force can yield a high load on stent members, and in particular on inner frame 305a and/or coupling arms 315a, 320a, and can even result in temporary atrial displacement of the inner frame 305a relative to the outer frame 310a and/or the native valve annulus. As should be understood, the heart beats dozens of times a minute, and a prosthetic heart valve may remain functional for years or decades. The cyclical stress put on the coupling arms 315a, 320a as the inner frame 305a is resisting atrial displacement during each heart beat can cause early fatigue of the stent, particularly at coupling arms 315a, 320a, and thus can decrease the overall fatigue life of the valve.

It should be understood that, although not shown in FIG. 3A, the inner frame 305a and/or outer frame 310a may include a skirt or cuff (e.g., tissue or fabric) along the luminal and/or abluminal surface(s) thereof that is substantially impermeable to blood flow to further seal the prosthetic heart valve 300a within the native valve annulus and to ensure that the only path for blood to flow is through the leaflets 330a when they are open.

FIG. 3B is a cross-sectional schematic diagram of a prosthetic heart valve 300b in the expanded condition and implanted relative to the atrium and ventricle. FIG. 5B is a perspective view of the prosthetic heart valve 300b of FIG. 3B.

As shown, prosthetic heart valve 300b may include a sealing feature 335b that covers a portion, but not the entirety, of the two-dimensional annular span S between coupling point 340b of the sealing feature 335b to inner frame 305a and a point O of outer frame 310a that is radially outward of the coupling point 340b. In the illustrated embodiment, the point O can be the same or similar height as coupling point 340b relative to inner frame 305a, and the coupling point 340b may be at an atrial-most point of the inner frame 305a and on an atrial side of anchoring point 325a. Stated another way, the sealing feature 335b can extend or span less than the entire radial distance between the coupling point 340b of the sealing feature 335b to inner frame 305a and a point O of outer frame 310a that is radially outward of the coupling point 340b. In this regard, the sealing feature 335b extends radially outward relative to the portion of outer frame 310a of least diameter. The sealing feature 335b may be formed of the same or similar material as that of sealing feature 335a. In another example, the coupling point 340b may be any point on the inner frame 305a on an atrial side of the anchoring point 325a such that the sealing feature 335b is positioned on an atrial side of the anchoring point 325a.

The sealing feature 335b can couple with both the inner frame 305a and the outer frame 310a. The sealing feature 335b may include a first portion 335b1 that couples with the inner frame 305a. The first portion 335b1 may couple with the inner frame 305a at any desired portion of the inner frame 305a, and in one example couples at a coupling point 340b that is the atrial-most point of the inner frame 305a. In this regard, the coupling point 340b is positioned radially inward, and on an atrial side, of an anchoring point 325a at which inner frame 305a couples with outer frame 310a. In another example, the coupling point 340b is any point on the inner frame 305a that is on an atrial side of anchoring point 325a, thereby providing the sealing feature 335b1 clearance with respect to the anchoring point 325a.

The sealing feature 335b may include a second portion 335b2 that couples with the outer frame 310a. The second portion 335b2 may couple with the outer frame 310a at any desired portion of the outer frame 310a, and in one example couples at a coupling point 345b that is between the atrial-most point of the outer frame 310a and a point 350a from which coupling arm 320a extends from outer frame 310a. In the illustrated example, the coupling point 345b is at or near the smallest inner diameter of the outer frame 310a when in the expanded condition. In another example, the second portion 335b2 may couple directly or indirectly with the coupling arm 320a. In both examples, the coupling point 345b may be positioned radially outward with respect to coupling point 340b. Further, in both examples, the coupling point 345b may be positioned both radially outward, and on a ventricular side, of an anchoring point 325a at which inner frame 305a couples with outer frame 310a.

The first portion 335b1 and the second portion 335b2 may be integrally formed with one another and such that the sealing feature 335b can be formed of a single material that is folded or otherwise contoured. In another example, the first portion 320b1 and the second portion 320b2 can be formed as separate members and coupled (e.g., sutured) with one another. In some examples, the first portion 335b1 and the second portion 335b2 can form an approximately right angle relative to one another.

The configuration of the sealing feature 335b results in the first portion 335b1 and thus sealing feature 335b extending and covering a portion, but not the entirety, of the annular span S, in a direction substantially orthogonal to the longitudinal axis of the prosthetic heart valve 300b, between coupling point 340b of the sealing feature 335b to inner frame 305a and a point O of outer frame 310a that is radially outward of the coupling point 340b, thereby defining a partially bounded three-dimensional annular pocket (represented P₁ in FIG. 3B). The partially bounded three-dimensional annular pocket P1 is bounded in part by inner frame 305a (and any cuff or skirt on the inner frame), first portion 335b1, second portion 335b2, and outer frame 310a (and any cuff or skirt on the outer frame). As depicted in FIG. 3B, the partially bounded three-dimensional annular pocket P1 may also be partially bounded by coupling arm 315a and coupling arm 320a at certain locations, as the coupling arm 315a and coupling arm 320a are arranged in a spaced apart manner around a circumference of the valve 300b. The pocket P1 is partially bounded insofar as the pocket P1 is relatively open on a ventricular or outflow side between the inner frame 305a and the portion of outer frame 310a of least diameter.

The portion of the annular span S not covered by sealing feature 335b between coupling point 340b and a point O of outer frame 310a that is radially outward of the coupling point 340b defines a partially bounded annular trough T₁. The partially bounded annular trough T₁ is positioned radially outward relative to pocket P1, at an inflow end portion of the outer frame 310a, and is relatively open on an atrial or inflow side of the valve 300b. The partially bounded annular trough T₁ can be partially bounded and defined in part by second portion 335b2 and outer frame 310a (and any cuff or skirt on the outer frame), but is otherwise unbounded relative to the atrium.

In FIG. 3B, the first portion 335b1 of sealing feature 335b extends radially outward, and is on an atrial side, relative to an anchoring point 325a at which inner frame 305a couples with outer frame 310a.

This configuration of sealing feature 335b results in a cross-sectional area A₂ that is less than the cross-sectional area A₁. This cross-sectional area A₂ is the combination of the cross-sectional surface area of the leaflets 330a (i.e., surface area of 2D projection of leaflets onto a plane that is orthogonal to direction of blood flow) in addition to the surface area of the first portion 335b1 of sealing feature 335b (the surface area of the first portion 335b1 being the same as the cross-sectional area of the sealing feature 335b). A resulting force on the inner frame 305a during ventricular systole is in the ventricle to atrium direction and is proportional, or equal, to the backpressure multiplied by the area A₂ against which the backpressure acts. The reduced cross-sectional area A₂, compared to cross-sectional area A₁, reduces a load on stent members and potentially resulting in an increase in fatigue life of the prosthetic heart valve 300b (as compared to prosthetic heart valve 300a). In other words, the area against which backpressure acts is smaller for prosthetic heart valve 300b than for prosthetic heart valve 300a. Notably, this reduction in fatigue does not reduce the ability of the sealing feature 335b providing a complete seal between the inner frame 305a and the outer frame 310a.

On the other hand, the sealing feature 335b creates the above-identified partially bounded annular trough T₁ near the inflow end of the outer frame 310a. During cyclic pumping, blood can stagnate in the partially bounded annular trough T₁, which can result in clotting and formation of a thrombus (represented as C in FIG. 3B). Such thrombus may be washed out by the flow of blood during the cyclic pumping, which may be undesirable, as the embolization of the thrombus may lead to a stroke or other infarction.

FIG. 3C is a cross-sectional schematic diagram of a prosthetic heart valve 300c in the expanded condition and implanted relative to the atrium and ventricle. FIG. 5C is a perspective view of the prosthetic heart valve 300c of FIG. 3C.

In this example, the sealing feature 335c may include a first portion 335c1 (that can be the same or similar to first portion 335b1), and a second portion 335c2 (that can be the same or similar to second portion 335b2). The prosthetic heart valve 300c may also include a third portion 335c3 (also referred to as embolic retention feature 335c3). In this example, a two-dimensional annular span (represented as S in FIG. 3C) between coupling point 340c of the sealing feature 335c to the inner frame 305a and coupling point 355c of the embolic retention feature 335c to the outer frame 310a is covered with a combination of sealing feature 335c (also referred to as a sealing member) and embolic retention feature 335c3. In this regard, the combination of sealing feature 335c and embolic retention feature 335c3 can extend or span an entire radial distance, in a direction substantially orthogonal to the longitudinal axis of the prosthetic heart valve 300c, between the coupling point 340b of the sealing feature 335c to inner frame 305a and coupling point 355c of the embolic retention feature 335c3 to outer frame 310a. The combination of sealing feature 335c and embolic retention feature 335c3 can be in the shape of a two-dimensional annulus, thereby defining an inner perimeter near the inner frame 305a and an outer perimeter near the outer frame 310a. A pocket closure is depicted in U.S. Pat. No. 9,597,181 to Christianson et al., filed Dec. 21, 2015, entitled Thrombus Management and Structural Compliance Features for Prosthetic Heart Valves, the entirety of which is incorporated by reference.

The sealing feature 335c can couple with the inner frame 305a and the embolic retention feature 335c3 can couple with the outer frame 310a. The sealing feature 335c can include the first portion 335c1 that couples with the inner frame 305a at a coupling point 340c (that can be the same or similar as coupling point 340b described above) and the second portion 335c2 that couples with the outer frame 310a at a coupling point 345c (that can be the same or similar as coupling point 345b described above). The embolic retention feature 335c3 may couple with outer frame 310a at a coupling point 355c. The coupling point 345c may be a distinct coupling location on the outer frame 310a relative to the coupling point 355c.

The embolic retention feature 335c3 may couple with the outer frame 310a at any desired portion of the outer frame 310a, and in one example couples at a coupling point 355c that is between the atrial-most point of the outer frame 310a and a point 350a from which coupling arm 320a extends from outer frame 310a. In this regard, the coupling point 355c may be positioned radially outward with respect to coupling point 340c and at a same or similar height as coupling point 340c relative to the longitudinal axis of inner frame 305a. The coupling point 355c may be positioned radially outward with respect to coupling point 345c and at a different height as coupling point 345c relative to the longitudinal axis of inner frame 305a. Further, the coupling point 355c may be positioned both radially outward, and on an atrial side, of an anchoring point 325a at which inner frame 305a couples with outer frame 310a.

The embolic retention feature 335c3 may be integrally formed with one or both of the first portion 335c1 and/or the second portion 335c2 such that the sealing portion 335c and embolic retention feature 335c3 can be formed of a single material that is folded. For example, the sealing feature 335c and embolic retention feature 335c3 may be formed as a single woven fabric, with the first portion 335c1 and the second portion 335c2 having tighter weaving (and thus less permeability) than embolic retention feature 335c3. In another example, the embolic retention feature 335c3 is coupled to either or both of the first portion 335c1 and/or the second portion 335c2. In some examples, the embolic retention feature 335c3 and the second portion 335c2 can form an approximately right angle relative to one another.

The embolic retention feature 335c3 can be formed at least in part, or in its entirety, of any suitable material that is sufficiently porous to allow blood, including particularly red blood cells, to enter pocket P2, but is not so porous as to allow undesirably large thrombi to leave the pocket P2, or to allow washout of thrombus formed in the pocket P2. For example, embolic retention feature 335c3 may be formed at least in part, or in its entirety, from a woven or knit polyester fabric with apertures less than 160 μm, and preferably between 90 μm and 120 μm. It is not necessary for the entirety of embolic retention feature 335c3 to be formed of the same material, with the same porosity. For example, some portions of embolic retention feature 335c3 may be formed of a less porous, or blood impermeable, material and other portions formed of material of the porosity range noted above. It is also contemplated that a portion of the outer frame 310a or the inner frame 305a may be formed with an aperture that communicates with pocket P2, covered by a closure formed of material having the desired porosity, thus providing another path by which blood may enter, but thrombi are prevented from leaving, pocket P2.

The embolic retention feature 335c3 may have a different permeability and/or porosity than either or both of the first portion 335c1 and the second portion 335c2. In one example, embolic retention feature 335c3 can have a higher permeability compared to one or both of the first portion 335c1 and the second portion 335c2. This may be achieved by any suitable mechanism, including controlling how tight the weave of the fabric is, if the embolic retention feature 335c3 is formed as a woven fabric.

The configuration of sealing feature 335c results in the combination of first portion 335c1 and embolic retention feature 335c3 extending and covering the entire annular span S, in a direction substantially orthogonal to the longitudinal axis of the prosthetic heart valve 300a, between the inner frame 305a and the outer frame 310a, thereby defining a partially bounded three-dimensional annular pocket (represented as P1 in FIG. 3C and the same or similar to pocket P1 in FIG. 3B). This configuration also defines a bounded second three-dimensional annular pocket (represented as P2 in FIG. 3C) bounded by outer frame 310a (and any cuff or skirt on the outer frame), embolic retention feature 335c3, and second portion 335c2 of sealing feature 335c. As depicted, the pocket P1 can be radially inward with respect to the pocket P2, with second portion 335c2 separating pocket P1 from pocket P2.

In FIG. 3C, the first portion 335c1 of sealing feature 335c extends radially outward, and is on an atrial side, relative to an anchoring point 325a at which inner frame 305a couples with outer frame 310a.

As in the example of FIG. 3B, this configuration of sealing feature 335c results in a cross-sectional area A₂ that is less than the cross-sectional area A₁ (and thus less total area against which backpressure may act). This cross-sectional area A₂ is the combination of the cross-sectional surface area of the leaflets 330a (i.e., surface area of 2D projection of leaflets onto a plane that is orthogonal to direction of blood flow) in addition to the cross-sectional area of the first portion 335c1 of sealing feature 335c. A resulting force on the inner frame 305a during ventricular systole is in the ventricle to atrium direction and is proportional, or equal, to the backpressure multiplied by the area A₂ against which the backpressure acts. The reduced cross-sectional area A₂ reduces a load on stent members and potentially resulting in an increase in fatigue life of the valve 300c (as compared to valve 300a). As should be understood, the resulting forces on prosthetic heart valve 300b and prosthetic heart valve 300c during ventricular systole are substantially similar, with the main difference being that prosthetic heart valve 300c also may be better suited to contain blood clots (represented as C in FIG. 3C) within the pocket P2. Advantageously, clot formation within pocket P2 can stiffen the valve over time, which can reduce the motion imposed on the frame from the native cardiac anatomy and can further increase the fatigue life of the stent.

Advantageously, the sealing features 335b and 335c provide a sealing feature between inner and outer valve assemblies, reduce force on stent members, therefore improving fatigue life of the stent. Further advantageously, the sealing feature 335c in combination with the embolic retention feature 335c3 provides a smooth transition from the native heart atrium to the prosthetic leaflets 330a and manages thrombus formation, preventing thrombosis or embolization of any thrombus trapped within the pocket Pa.

FIG. 3D is a cross-sectional schematic diagram of a prosthetic heart valve 300d in the expanded condition and implanted relative to the atrium and ventricle.

In this example, the heart valve 300d can include a first portion 335d1 (also referred to as embolic retention feature 335d1) and sealing feature 335d2.

The embolic retention feature 335d1 can extend or span an entire radial distance, in a direction substantially orthogonal to the longitudinal axis of the prosthetic heart valve 300d, between coupling point 340d of inner frame 305a and coupling point 345d of outer frame 310a. The embolic retention feature 335d1 can be in the shape of a two-dimensional annulus, thereby defining an inner perimeter near the inner frame 305a and an outer perimeter near the outer frame 310a.

The embolic retention feature 335d1 can couple with both the inner frame 305a and the outer frame 310a. The embolic retention feature 335d1 may couple with the inner frame 305a at any desired portion of the inner frame 305a, and in one example couples at a coupling point 340d that is the atrial-most point of the inner frame 305a. In this regard, the coupling point 340d is positioned radially inward, and on an atrial side, of an anchoring point 325a at which inner frame 305a couples with outer frame 310a. In another example, the coupling point 340d is any point on the inner frame 305a that is on an atrial side of anchoring point 325a, thereby providing the embolic retention feature 335d1 clearance with respect to the anchoring point 325a.

The embolic retention feature 335d1 may couple with the outer frame 310a at any desired portion of the outer frame 310a, and in one example couples at a coupling point 345d that is between the atrial-most point of the outer frame 310a and a point 350a from which coupling arm 325a extends inward from outer frame 310a. In this regard, the coupling point 345d may be positioned radially outward with respect to coupling point 340d. Further, the coupling point 345d may be positioned both radially outward, and on an atrial side, of an anchoring point 325a at which inner frame 305a couples with outer frame 310a. In the illustrated embodiment, embolic retention feature 335d1 extends in a direction that is generally orthogonal to the direction of blood flow, but in other embodiments, the embolic retention feature 335d1 may extend at an oblique angle relative to the direction of blood flow.

The sealing feature 335d2 may also couple with both the inner frame 305a and the outer frame 310a. The sealing feature 335d2 may couple with the inner frame 305a at any desired portion of the inner frame 305a between the ventricular-most point of the inner frame 305a and a point 360d from which coupling arm 315a extends from inner frame 305a. In one example, the sealing feature 335d2 couples at a coupling point 350d that is radially inward of a central waist W region (e.g., a portion of minimum radius) of outer frame 310a. In this regard, the coupling point 350d is positioned radially inward, and on a ventricular side, of an anchoring point 325a at which inner frame 305a couples with outer frame 310a.

The sealing feature 335d2 may also couple with the outer frame 310a at any desired portion of the outer frame 310a, and in one example couples at a coupling point 355d that is between the ventricular-most point of the outer frame 310a and a point 350a from which coupling arm 325a extends inward from outer frame 310a. In this regard, the coupling point 355d may be positioned radially outward with respect to coupling point 350d. Further, the coupling point 355d may be positioned both radially outward, and on a ventricular side, of an anchoring point 325a at which inner frame 305a couples with outer frame 310a. In the illustrated embodiment, the sealing feature 335d2 extends in a direction that is generally orthogonal to the direction of blood flow, but in other embodiments, the sealing feature 335d2 may extend at an oblique angle relative to the direction of blood flow.

This configuration results in embolic retention feature 335d1 extending and covering the entire two-dimensional annular span S between the coupling point 340d at inner frame 305a and the coupling point 345d at outer frame 310a, with the sealing feature 335d2 extending and covering a portion, but not the entirety, of the annular span S, in a direction substantially orthogonal to the longitudinal axis of the prosthetic heart valve 300d, between coupling point 350d of the sealing feature 335d2 to inner frame 305a and coupling point 355d to outer frame 310a that is radially outward of the coupling point 340d, thereby defining a three-dimensional annular pocket (represented P3 in FIG. 3D).

The three-dimensional annular pocket P3 is bounded in part by inner frame 305a (and any cuff or skirt on the inner frame), embolic retention feature 335d1, sealing feature 335d2, and outer frame 310a (and any cuff or skirt on the outer frame). As depicted in FIG. 3D, the bounded three-dimensional annular pocket P3 may also be partially bounded by coupling arm 315a and coupling arm 320a at certain locations, as the coupling arm 315a and coupling arm 320a are arranged in a spaced apart manner around a circumference of the valve 300d.

The embolic retention feature 335d1 can be formed at least in part, or in its entirety, of any suitable material that is sufficiently porous to allow blood, including particularly red blood cells, to enter pocket P3, but is not so porous as to allow undesirably large thrombi to leave the pocket P3, or to allow washout of thrombus formed in the pocket P3. For example, embolic retention feature 335d1 may be formed at least in part, or in its entirety, from a woven or knit polyester fabric with apertures less than 160 μm, and preferably between 90 μm and 120 μm. It is not necessary for the entirety of embolic retention feature 335d1 to be formed of the same material, with the same porosity. For example, some portions of embolic retention feature 335d1 may be formed of a less porous, or blood impermeable, material and other portions formed of material of the porosity range noted above. It is also contemplated that a portion of the outer frame 310a or the inner frame 305a may be formed with an aperture that communicates with pocket P3, covered by a closure formed of material having the desired porosity, thus providing another path by which blood may enter, but thrombi are prevented from leaving, pocket P3.

The sealing feature 335d2 can be formed at least in part, or in its entirety, of any suitable material that is substantially impermeable to blood and thereby prevents blood flow across the prosthetic heart valve 300d and through the sealing feature 335d2 when the prosthetic leaflets 330a are in the closed or coapted condition. The sealing feature 335d2 can be formed of a tissue or a fabric, including synthetic fabrics, such as PET, PTFE, etc.

The embolic retention feature 335d1 may have a different permeability and/or porosity than the sealing feature 335d2. In one example, embolic retention feature 335d1 can have a higher permeability compared to sealing feature 335d2. This may be achieved by any suitable mechanism, including controlling how tight the weave of the fabric is, if the embolic retention feature 335d1 is formed as a woven fabric.

This configuration of sealing feature 335d2 results in a cross-sectional area A₃ that is less than the cross-sectional area A₁. This cross-sectional area A₃ is the combination of the cross-sectional surface area of the leaflets 330a (i.e., surface area of 2D projection of leaflets onto a plane that is orthogonal to direction of blood flow) in addition to the surface area of the sealing feature 335d2. A resulting force on the inner frame 305a during ventricular systole is in the ventricle to atrium direction and is proportional, or equal, to the backpressure multiplied by the area A₃ against which the backpressure acts. The reduced cross-sectional area A₃, compared to cross-sectional area A₁, reduces a load on stent members and potentially resulting in an increase in fatigue life of the prosthetic heart valve 300d (as compared to prosthetic heart valve 300a). In other words, the area against which backpressure acts is smaller for prosthetic heart valve 300d than for prosthetic heart valve 300a. Notably, this reduction in fatigue does not reduce the ability of the sealing feature 335d2 providing a complete seal between the inner frame 305a and the outer frame 310a.

FIG. 3E is a cross-sectional schematic diagram of a prosthetic heart valve 300e in the expanded condition and implanted relative to the atrium and ventricle.

In this example, the heart valve 300e can include a first portion 335e1 (also referred to as embolic retention feature 335e1) and a sealing feature 335e2. The embolic retention feature 335e1 can be the same or similar to embolic retention feature 335d1 depicted in FIG. 3D and can couple with both the inner frame 305a at coupling point 340e and with the outer frame 310a at coupling point 355e.

The sealing feature 335e2 may also couple with both the inner frame 305a and the outer frame 310a. The sealing feature 335e2 may couple with the inner frame 305a at any desired portion of the inner frame 305a, and in one example couples at a coupling point 340e that is the atrial-most point of the inner frame 305a. In this regard, the coupling point 340e is positioned radially inward, and on an atrial side, of an anchoring point 325a at which inner frame 305a couples with outer frame 310a. In another example, the coupling point 340e is any point on the inner frame 305a that is on an atrial side of anchoring point 325a, thereby providing the sealing feature 335e2 clearance with respect to the anchoring point 325a.

Both the sealing feature 335e2 and embolic retention feature 335e1 may couple to the inner frame 305a at coupling point 340e such that the sealing feature 335e2 and embolic retention feature 335e1 may be coupled to one another or the sealing feature 335e2 and embolic retention feature 335e1 may be coupled independently to inner frame 305a.

The sealing feature 335e2 and embolic retention feature 335e1 may be integrally formed with one another and such that the they can be formed of a single material that is folded or otherwise contoured. In another example, the sealing feature 335e2 and embolic retention feature 335e1 can be formed as separate members and coupled (e.g., sutured) with one another.

The sealing feature 335e2 may also couple with the outer frame 310a at any desired portion of the outer frame 310a, and in one example couples at a coupling point 345e that is between the atrial-most point of the outer frame 310a and a point 350a from which coupling arm 320a extends from outer frame 310a. In the illustrated example, the coupling point 345e is at or near the smallest inner diameter of the outer frame 310a when in the expanded condition.

The coupling point 345e may be positioned radially outward with respect to coupling point 340e and radially inward with respect to coupling point 355e. Further, the coupling point 345e may be positioned both radially outward, and on a ventricular side, of an anchoring point 325a at which inner frame 305a couples with outer frame 310a. In one example, the sealing feature 335e2 may also couple directly or indirectly with the anchoring point 325a.

In the illustrated embodiment, the sealing feature 335e2 extends at an oblique angle relative to the direction of blood flow. In this regard, the sealing feature 335e2 couples to the inner frame 305a at coupling point 340e that is on an atrial-side of anchoring point 325a and couples to the outer frame 310a a coupling point 345e that is on a ventricular side of anchoring point 325a, with the coupling point 345e being on an atrial side of point 350a from which coupling arm 320a extends from outer frame 310a. Stated another way, the coupling point 345e can be on a ventricular side of coupling point 340e.

This configuration is such that embolic retention feature 335e1 can extend and cover the entire two-dimensional annular span S between the coupling point 340e at inner frame 305a and the coupling point 355e at outer frame 310a. The sealing feature 335e2 can extend and cover a portion, but not the entirety, of the annular span S, in a direction oblique to the longitudinal axis of the prosthetic heart valve 300d, between coupling point 340e of the sealing feature 335e2 to inner frame 305a and coupling point 345e to outer frame 310a that is radially outward of and in a ventricular direction relative to the coupling point 340e, thereby defining a three-dimensional annular pocket (represented P4 in FIG. 3E).

The three-dimensional annular pocket P4 is bounded in part by inner frame 305a (and any cuff or skirt on the inner frame), embolic retention feature 335e1, sealing feature 335e2, and outer frame 310a (and any cuff or skirt on the outer frame).

The embolic retention feature 335e1 can be formed at least in part, or in its entirety, of any suitable material that is sufficiently porous to allow blood, including particularly red blood cells, to enter pocket P4, but is not so porous as to allow undesirably large thrombi to leave the pocket P4, or to allow washout of thrombus formed in the pocket P4. For example, embolic retention feature 335e1 may be formed at least in part, or in its entirety, from a woven or knit polyester fabric with apertures less than 160 μm, and preferably between 90 μm and 120 μm. It is not necessary for the entirety of embolic retention feature 335e1 to be formed of the same material, with the same porosity. For example, some portions of embolic retention feature 335e1 may be formed of a less porous, or blood impermeable, material and other portions formed of material of the porosity range noted above. It is also contemplated that a portion of the outer frame 310a or the inner frame 305a may be formed with an aperture that communicates with pocket P4, covered by a closure formed of material having the desired porosity, thus providing another path by which blood may enter, but thrombi are prevented from leaving, pocket P4.

The sealing feature 335e2 can be formed at least in part, or in its entirety, of any suitable material that is substantially impermeable to blood and thereby prevents blood flow across the prosthetic heart valve 300e and through the sealing feature 335e2 when the prosthetic leaflets 330a are in the closed or coapted condition. The sealing feature 335e2 can be formed of a tissue or a fabric, including synthetic fabrics, such as PET, PTFE, etc.

The embolic retention feature 335e1 may have a different permeability and/or porosity than the sealing feature 335e2. In one example, embolic retention feature 335e1 can have a higher permeability compared to sealing feature 335e2. This may be achieved by any suitable mechanism, including controlling how tight the weave of the fabric is, if the embolic retention feature 335e1 is formed as a woven fabric.

This configuration of sealing feature 335e2 results in a cross-sectional area A₄ that is less than the cross-sectional area A₁. This cross-sectional area A₄ is the combination of the cross-sectional surface area of the leaflets 330a (i.e., surface area of 2D projection of leaflets onto a plane that is orthogonal to direction of blood flow) in addition to the cross-sectional surface area of the sealing feature 335e2 (i.e., surface area of 2D projection of sealing feature 335e2 onto a plane that is orthogonal to direction of blood flow). A resulting force on the inner frame 305a during ventricular systole is in the ventricle to atrium direction and is proportional, or equal, to the backpressure multiplied by the area A₄ against which the backpressure acts. The reduced cross-sectional area A₄, compared to cross-sectional area A₁, reduces a load on stent members and potentially resulting in an increase in fatigue life of the prosthetic heart valve 300e (as compared to prosthetic heart valve 300a). In other words, the area against which backpressure acts is smaller for prosthetic heart valve 300e than for prosthetic heart valve 300a. Notably, this reduction in fatigue does not reduce the ability of the sealing feature 335e2 providing a complete seal between the inner frame 305a and the outer frame 310a.

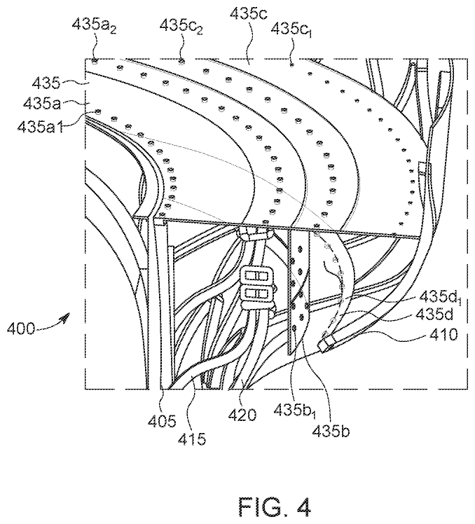

FIG. 4 illustrates a partial cross-sectional view of a prosthetic valve 400 in the expanded condition. Similar to the example described above with respect to FIG. 3C, the prosthetic valve 400 can include an inner frame 405, an outer frame 410, leaflets (not shown), coupling arms 415 and 420, and a sealing feature 435. The sealing feature 435 may include a first portion 435a and a second portion 435b. The prosthetic valve 400 may also include an embolic retention feature 435c. The sealing feature 435 may also include a fourth portion 435d. Although provided with different part numbers, prosthetic heart valve 400 may indeed be identical to prosthetic heart valve 300c.

The fourth portion 435d may be integrally formed with the second portion 435b and may extend substantially along a curvilinear profile defined by outer frame 410. In this regard, the fourth portion 435d can form an acute angle with respect to second portion as measured from an atrial side. The fourth portion 435d may include a plurality of suture holes 435d1 positioned radially and may be used to suture the fourth portion 435d to the outer frame 410 or a skirt (not shown) attached to the outer frame 410.

The first portion 435a may include first suture holes 435al and/or second suture holes 435a2. The first suture holes 435al may be positioned radially inward relative to the second suture holes 435a2 such that the first suture holes are at or near inner frame 405 and the second suture holes 435a2 are at or near a junction among first portion 435a, second portion 435b, and/or embolic retention feature 435c. The first suture holes 435al may be used to suture the sealing feature 435 directly to inner frame 405 or to a skirt (not shown) attached to the inner frame 405.

The embolic retention feature 435c may include first suture holes 435c1 and/or second suture holes 435c2. The first suture holes 435c1 may be positioned radially outward relative to the second suture holes 435c2 such that the first suture holes 435c1 are at or near outer frame 410 and the second suture holes 435c2 are at or near a junction among first portion 435a, second portion 435b, and/or embolic retention feature 435c. The first suture holes 435c1 may be used to suture the sealing feature 435 directly to outer frame 410 or to a skirt (not shown) attached to the inner frame 410. The second suture holes 435c2 may be used, along with second suture holes 435a2 and suture holes 435b1 of second portion 435b, to suture the first portion 435a, second portion 435b, and/or embolic retention feature 435c to one another. Any of the examples described above with respect to FIGS. 3B and/or 3C may incorporate a fourth portion to assist in stability of the overall sealing feature.

The various suture holes may be created by any suitable mechanism, including a laser. The suture holes may assist in providing a more repeatable suture pattern, as well as making the manufacturing process significantly simpler and less time-consuming.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
an inner frame having a longitudinal axis;
a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis;
an outer frame connected to the inner frame; and
a sealing feature coupled to the inner frame at a coupling point and extending partially, but not completely, between the coupling point and a point of the outer frame that is radially outward of the coupling point in a direction substantially orthogonal to the longitudinal axis, and wherein in an implanted condition, the sealing feature is positioned such that backpressure from ventricular systole acts on the sealing feature.

2. The prosthetic heart valve of claim 1, wherein the sealing feature comprises a first portion and a second portion.

3. The prosthetic heart valve of claim 2, wherein the first portion, the second portion, the inner frame, and the outer frame define a partially bounded first pocket that is open and faces a ventricular side of the prosthetic valve.

4. The prosthetic heart valve of claim 2, wherein the first portion couples with the inner frame at the coupling point, and the second portion couples with the outer frame at a second coupling point.

5. The prosthetic heart valve of claim 4, wherein the inner frame comprises a first coupling arm extending radially outward of the longitudinal axis and the outer frame comprises a second coupling arm extending radially inward toward the longitudinal axis such that the first coupling arm and the second coupling arm meet and are coupled together at an anchoring point.

6. The prosthetic heart valve of claim 5, wherein the second coupling point is positioned between an atrial-most point of the outer frame and a point at which the second coupling arm extends from the outer frame.

7. The prosthetic heart valve of claim 2, wherein the first portion and the second portion each define suture holes for suturing the respective portions.

8. The prosthetic heart valve of claim 2, wherein the sealing feature further comprises a fourth portion extending radially outward from the second portion, the fourth portion defining suture holes.

9. The prosthetic heart valve of claim 1, further comprising an embolic retention feature that couples with the outer frame such that a combination of the sealing feature and the embolic retention feature extend completely between the coupling point and the point of the outer frame that is radially outward of the coupling point in the direction substantially orthogonal to the longitudinal axis.

10. A prosthetic heart valve, comprising:
an inner frame having a longitudinal axis;
a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis;
an outer frame connected to the inner frame;
a sealing feature including a first portion coupled to the inner frame at a first coupling point, a second portion coupled to the outer frame at a second coupling point; and
an embolic retention feature coupled to the outer frame at a third coupling point, wherein the embolic retention feature has a permeability that is greater than at least one of the first portion or the second portion.

11. The prosthetic heart valve of claim 10, wherein the first portion, the second portion, the inner frame, and the outer frame define a partially bounded first pocket that is open and faces a ventricular side of the prosthetic valve, and the second portion, embolic retention feature, and outer frame define a bounded pocket configured to retain a blood clot therein.

12. The prosthetic heart valve of claim 10, wherein the embolic retention feature has a permeability that is greater than both of the first portion and the second portion.

13. The prosthetic heart valve of claim 10, wherein the inner frame comprises a first coupling arm extending radially outward of the longitudinal axis and the outer frame comprises a second coupling arm extending radially inward toward the longitudinal axis such that the first coupling arm and the second coupling arm meet and are coupled together at an anchoring point.

14. The prosthetic heart valve of claim 13, wherein the second coupling point is on a ventricular side of the anchoring point and the third coupling point is on an atrial side of the anchoring point.

15. The prosthetic heart valve of claim 11, wherein the third coupling point is positioned radially outward relative to the second coupling point.

16. A prosthetic heart valve, comprising:
an inner frame having a longitudinal axis;
a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis;
an outer frame;
a sealing feature coupled to the inner frame at a first coupling point and extending radially outward and coupling to the outer frame at a second coupling point; and
an embolic retention feature coupled to the outer frame at a third coupling point that is distinct from the second coupling point such that a partially bounded pocket is defined and a bounded pocket is defined, wherein the bounded pocket is configured to retain a blood clot therein.

17. The prosthetic heart valve of claim 16, wherein the sealing feature comprises a first portion and a second portion.

18. The prosthetic heart valve of claim 17, wherein a combination of the first portion and the embolic retention feature spans an entire annular span between the first coupling point and the third coupling point.

19. The prosthetic heart valve of claim 17, wherein the embolic retention feature has a greater permeability than both of the first portion and the second portion.

20. The prosthetic heart valve of claim 17, wherein the partially bounded pocket is defined in part by the first portion and the second portion such that the partially bounded pocket is radially inward relative to the bounded pocket.
